(19) United States
(12) Patent Application Publication
(10) Pub. No.: US 2023/0092662 A1
(43) Pub. Date: Mar. 23, 2023

(54) CISPLATIN NANOPARTICLE COMPOSITION, METHOD FOR THE PREPARATION THEREOF

(71) Applicant: Sarasija Suresh, Bengaluru (IN)

(72) Inventors: Sarasija Suresh, Bangalore (IN); Vishal Uchila Shishir Rao, Bangalore (IN); S. Narasimha Murthy, Bangalore (IN); H.N. Shivakumar, Bangalore (IN)

(73) Assignees: Sarasija Suresh, Bengaluru (IN); Vishal Uchila Shishir Rao, Bangalore (IN)

(21) Appl. No.: 17/796,088

(22) PCT Filed: Nov. 16, 2018
(86) PCT No.: PCT/IB2018/059027

§ 371 Date: Jul. 28, 2022

(30) Foreign Application Priority Data
Nov. 17, 2017 (IN) 201741041197

(51) Int. Cl. A61K 9/51; A61K 33/243

(52) U.S. Cl. CPC A61K 9/5192; A61K 33/243; A61K 9/5123

(57) ABSTRACT

The present disclosure relates to cisplatin nanoparticle compositions and a method for the preparation thereof. A phospholipid complex of cisplatin is prepared for increased absorption, followed by the phospholipid complex is converted into lipid nanoparticles by choosing appropriate solvents, incorporation of lipids, stabilizers under optimum conditions of agitation, temperature and solvent evaporated under reduced pressure. The incorporation of lipids and stabilizers for the formulation of nanoparticles based cisplatin leads to the formation of micelles and mixed micelles that enhance the cisplatin absorption into systemic circulation because of the nano size and by lymphatic transport. The nanoparticle based composition of cisplatin that is administered orally. It is safe, effective, convenient and affordable to the patient.

### DESCRIPTION OF THE INVENTION

### Technical Field of the Invention

The present invention generally relates to the field of composition for cancer care. More particularly, the invention relates to cisplatin nanoparticle composition that is delivered orally. The invention also related to a method of preparing the composition.

### BACKGROUND OF THE INVENTION

Cancer figures among the leading cause of morbidity and mortality worldwide. India is likely to have 17.3 lakh new cases and 8.8 lakh deaths due to cancer by 2020. It is reported that one in eight Indians is likely to develop cancer in their lifetime. The standard treatment for cancer continues to be radiation, surgery and chemotherapy. Toxicity, resistance, hospitalization, cost, pain and patient inconvenience continue to be challenges in effective anticancer therapy.

Cancer chemotherapy has been an ever-expanding area of scientific endeavor, and has been a critical component of cancer treatment along with surgery and radiation therapy. Where chemotherapy was once accepted only as a means to extend survival time for those patients diagnosed as incurable by surgery and radiation therapy, it is now a recognized modality of treatment in nearly all of the more than two thousand variations of cancer.

Modem cancer chemotherapy typically involves a combination of two or three different drugs, and the advances in technology and medical knowledge have greatly improved a patient's chances of recovery in many forms of cancer. The role of antineoplastic agents in cancer therapy varies widely depending upon the form of cancer. For example, chemotherapy is often the primary course of therapy in cancers of the ovary, testis, breast, bladder, and others, in leukemias and lymphomas, and is generally employed in combination with radiation therapy in the treatment of a large number of sarcomas, melanomas, myelomas, and others. In contrast, chemotherapy is often used only as a last resort or as a palliative treatment for most solid tumors, such as carcinomas of the pancreas and lung. There are exceptions within each class of tumor or other neoplasm.

Cisplatin is an anticancer agent known for its effectiveness most widely prescribed platinum compounds has become an invaluable component of therapy for solid tumors such as head and neck, testicular, lung, ovarian, cervical and bladder cancers. However, significant side effects of cisplatin are also observed when it is administered intravenously, in particular nephrotoxicity, gastrointestinal toxicity (nausea, vomiting), neurotoxicity and moderate myelo-suppression. The ability to deliver cisplatin orally would allow greater flexibility, convenience and affordability to the patient.

Currently there are no cisplatin formulations available that can be administered orally and could be safe, effective, convenient and affordable to the patient.

Objective of the Invention

It is therefore an object of the present invention to produce nanoparticles based composition comprising cisplatin.

It is another object of the present invention to produce a formulation comprising the nanoparticles composition that contains cisplatin for oral delivery.

It is yet another object of the present invention to provide a safe, effective and affordable formulation comprising nanoparticles composition containing cisplatin with reduced dose-related toxicity and the composition is.

It is still another object of the present invention to provide a formulation comprising the nanoparticles composition containing cisplatin which can overcome antineoplastic resistance.

It is still another object of the present invention to provide an oral cisplatin formulation for metronomic cancer therapy for increased activity, reduced toxicity and adverse effects

### SUMMARY OF THE INVENTION

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

The present invention overcomes the drawbacks in the prior art and provides a method for preparing a cisplatin nanoparticle composition for oral administration comprising dissolving predetermined amount of cisplatin and phospholipid in a suitable solvent, refluxing the above mixture under optimized conditions of temperature, duration and stirring rate until complexation is complete, removing cisplatin phospholipid complex by non-solvent addition, drying the cisplatin phospholipid complex under vacuum, dissolving the cisplatin phospholipid complex in fatty acids and lipids at temperatures ranging from 50-70 C, adding hot lipid solution to aqueous solution of predetermined amount of surfactant, stirring under optimized conditions of temperature and duration and obtaining a cisplatin lipid nanoparticles.

The cisplatin and phospholipid are added in a molar concentration of 0.5:0.5 to 3. The fatty acids and lipids in which cisplatin phospholipid complex is dissolved are stearic acid, palmitic acid and oleic acid. The surfactant in which hot lipid solution is added is of quantity 0.5 to 2.5%.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 shows a flow chart in accordance to one or more embodiment of the present invention.

FIG. 2 shows a graph of cisplatin in accordance to one or more embodiment of the present invention.

FIG. 3 shows a permeability study of cisplatin in accordance to one or more embodiment of the present invention.

FIG. 4 shows a graph of cisplatin in accordance to one or more embodiment of the present invention.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

In order to more clearly and concisely describe and point out the subject matter of the claimed invention, the following definitions are provided for specific terms, which are used in the following written description.

The term “Nanoparticle”, means a particle that has a size of nanometers or tens of nanometers as the context requires.

The term “Permeability”, means a property of a material that lets fluids to diffuse through it to another medium without being chemically or physically affected, as the context requires.

The term “Absorption”, means a process in which one substance permeates another, a fluid permeates or is dissolved by a liquid or solid, as the context requires.

The term “Phospholipid complex”, means a chemical that is attached to a lipid that contains phosphorus, as the context requires.

The term “MTT assay” means a colorimetric assay for assessing cell metabolic activity, as the context requires.

The present invention overcomes the drawbacks of the existing state of the art technologies by providing a nanoparticle based composition of cisplatin that is administered orally and is safe, effective, convenient and affordable to the patients.

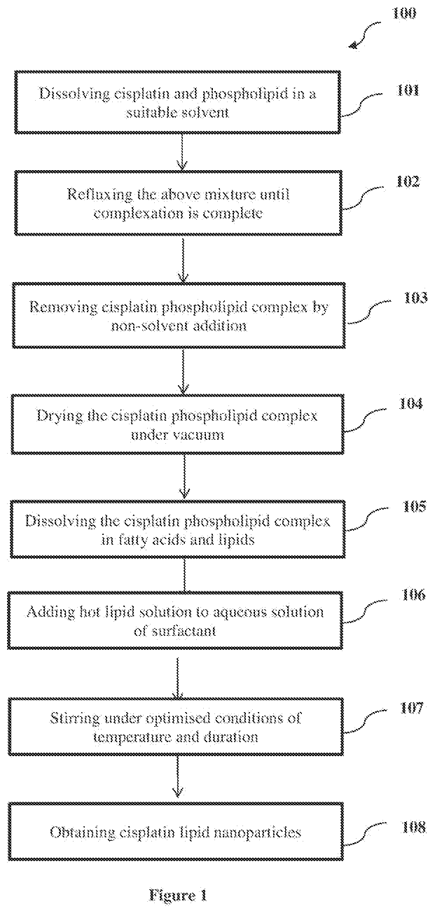

FIG. 1 illustrates a flowchart depicting a method of preparation of nanoparticle based composition containing cisplatin in accordance with one or more embodiment of the present invention. The phospholipid complex of cisplatin is prepared by non-solvent precipitation method. The lipid nanoparticles of cisplatin are formed by hot homogenization method. The phospholipid complex is converted into lipid nanoparticles by choosing appropriate solvents, incorporation of lipids, stabilizers under optimum conditions of agitation, temperature and solvent evaporated under reduced pressure. The incorporation of lipids and stabilizers for the formulation of nanoparticles based cisplatin leads to the formation of micelles and mixed micelles that enhance the cisplatin absorption into systemic circulation because on the nano size and by lymphatic transport. As shown in FIG. 1 suitable molar concentrations of cisplatin and phospholipid (0.5:0.5 to 3) are dissolved in a suitable solvent (101) and refluxed until complexation is complete under optimized conditions of temperature, duration and stirring rate (102). At step (103), the formed complex is removed by non-solvent addition and dried under vacuum (104). At step (105), the cisplatin phospholipid complex is dissolved in fatty acids and lipids such as stearic acid, palmitic acid, oleic acid at temperatures ranging from 50-70 C. At step (106), the hot lipid solution is added to aqueous solution of surfactant (0.5 to 2.5%) and stirred under optimized conditions of temperature and duration (107). The product obtained is separated and dried to obtain cisplatin lipid nanoparticles (108).

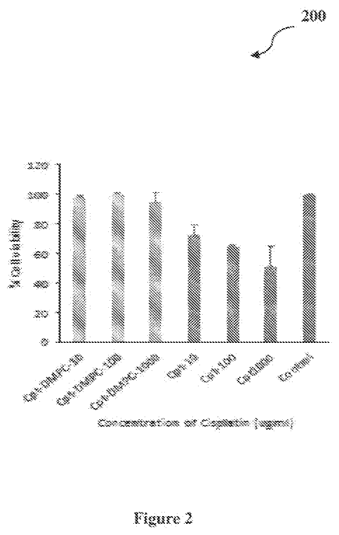

FIG. 2 shows the effect of phospholipid complex versus pure cisplatin on HEK cell line by MTT assay. Enhanced cell viability was observed in the presence of the phospholipid complex as compared to pure cisplatin, indicating the enhanced safety of the complex.

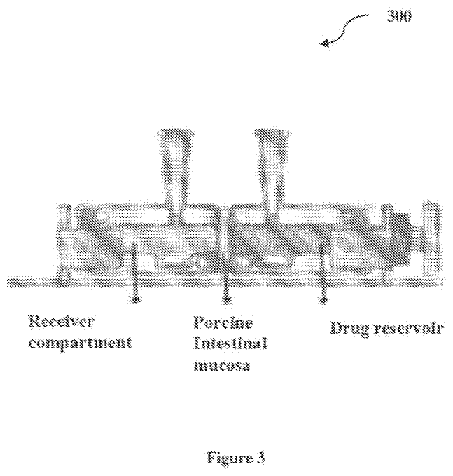

FIG. 3 shows the permeability of phospholipid complex of cisplatin, which is the main component of Nanocisplatin versus the pure cisplatin. It was studied through porcine intestinal mucosa. Considerable enhancement in the absorption of cisplatin was observed from the phospholipid complex.

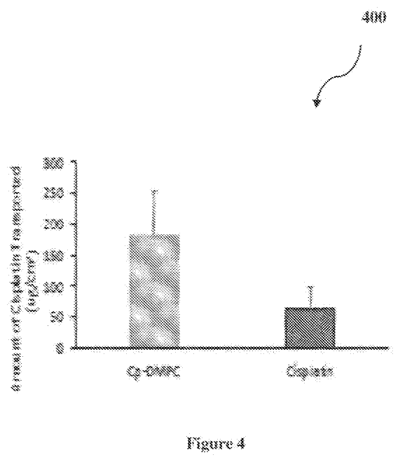

FIG. 4 shows the effect of phospholipid complex versus pure cisplatin on HEK cell line. The cisplatin complexed with phospholipid showed better transport through porcine intestinal mucosa.

Owing to the nano size of the cisplatin formulation, it is highly stable, high carrier capacity, feasibility of incorporation of both hydrophilic and hydrophobic substances, and feasibility of variable routes of administration, including oral application and inhalation.

While at least one exemplary embodiment has been presented in the foregoing summary and detailed description, it should be appreciated that a vast number of variations exist. It should also be appreciated that the exemplary embodiment or exemplary embodiments are only examples, and are not intended to limit the scope, applicability, or configuration in any way. Rather, the foregoing summary and detailed description will provide those skilled in the art with a convenient road map for implementing an exemplary embodiment, it being understood that various changes may be made in the function and arrangement of elements described in an exemplary embodiment without departing from the scope as set forth in the appended claims and their legal equivalents.

I claim:

1. A method for preparing a cisplatin nanoparticle composition for oral administration, the method comprising the steps of:
a) dissolving predetermined amount of cisplatin and phospholipid in a suitable solvent;
b) refluxing the above mixture under optimized conditions of temperature, duration and stirring rate until complexation is complete;
c) removing cisplatin phospholipid complex from refluxed mixture by non-solvent addition;
d) drying the cisplatin phospholipid complex under vacuum;
e) dissolving the cisplatin phospholipid complex in fatty acids and lipids at temperatures ranging from 50-70 C;
f) adding hot lipid solution to aqueous solution of predetermined amount of surfactant;
g) stirring under optimised conditions of temperature and duration;
h) obtaining cisplatin lipid nanoparticles.

2. The method as claimed in claim 1, wherein the cisplatin and phospholipid are added in a molar concentration of 0.5:0.5 to 3.

3. The method as claimed in claim 1, wherein the fatty acids and lipids in which cisplatin phospholipid complex is dissolved are stearic acid, palmitic acid and oleic acid.

4. The method as claimed in claim 1, wherein the surfactant in which hot lipid solution is added is of quantity 0.5 to 2.5%.

5. The method as claimed in claim 1, wherein said cisplatin composition when used in metronomic doses increases efficacy of anticancer therapy.
